(19) United States
(12) Patent Application Publication
(10) Pub. No.: US 2023/0256357 A1
(43) Pub. Date: Aug. 17, 2023

(54) DEVICE FOR SOLID/LIQUID EXTRACTION BY RADIAL ULTRASONIC IRRADIATION, AND ASSOCIATED EXTRACTION METHOD

(71) Applicant: UNIVERSITÉ SAVOIE MONT BLANC, CHAMBERY (FR)

(72) Inventors: Grégory CHATEL, BRENS (FR); Micheline DRAYE, CHAMBERY (FR); Romain DUWALD, CHAMBERY (FR); Philippe FANGET, SAINT GENIX LES VILLAGES (FR); Christine PIOT, BARBY (FR)

(21) Appl. No.: 18/009,679

(22) PCT Filed: Jun. 11, 2021
(86) PCT No.: PCT/EP2021/065820

§ 371 Date: Dec. 9, 2022

(30) Foreign Application Priority Data
Jun. 12, 2020 (FR) 2006171

(51) Int. Cl. B01D 11/02; C07C 37/82; C07H 15/207; C07C 46/10

(52) U.S. Cl. CPC B01D 11/0265; B01D 11/0257; B01D 11/0223; C07C 37/82; C07H 15/207; C07C 46/10

(57) ABSTRACT

Disclosed is a continuous solid/liquid extraction device including at least, from upstream to downstream: a mixer including an inlet and an outlet; an extraction coil including an inlet and an outlet, the inlet of the coil being connected to the outlet of the mixer; and a phase separator connected to the outlet of the coil. The same coil includes an ultrasonic probe with radial irradiation placed in the center of the coil.

### CROSS-REFERENCE TO RELATED APPLICATION(S)

This application is the U.S. national phase of International Application No. PCT/EP2021/065820 filed Jun. 11, 2021 which designated the U.S. and claims priority to FR 2006171 filed Jun. 12, 2020, the entire contents of each of which are hereby incorporated by reference.

### TECHNICAL FIELD

The present invention relates to a device for solid/liquid extraction.

### BACKGROUND

Among conventional solid/liquid extraction devices, in particular for obtaining molecules of interest from ground material of plant origin, it is common to use ultrasonic apparatuses. Optimized devices involving ultrasound can limit the energy consumption associated with prolonged heating and eliminate the use of potentially toxic organic solvents. They also make it possible to operate at room temperature and thus limit the degradation of certain sensitive organic molecules.

For this purpose, ultrasonic tanks are commonly used in industry for treating large volumes and for working on batches. Nevertheless, such non-optimized devices have many drawbacks for the extraction, such as the impossibility of controlling the rise in temperature generated by ultrasound which can lead to the degradation of certain thermosensitive molecules.

Moreover, currently, the extraction in ultrasonic tanks can only be performed on batches. Such a way of working requires emptying the tanks completely after each batch processed, cleaning the tanks and filtering the medium on ancillary equipment. Hence, steps of transfer and transport of the mixture must be added, entailing material losses and significant handling times.

### SUMMARY OF THE INVENTION

One of the goals of the invention is to propose a compact continuous solid/liquid extraction device, allowing for the rapid and efficient extraction of molecules of interest from solid material suspended in a liquid solvent, while maintaining the fluidity and the homogeneity of the suspension and making it possible to subsequently use one filtration step.

To this end, the subject material of the invention is a continuous solid/liquid extraction device comprising, from upstream to downstream, at least:

a mixer comprising an inlet and an outlet, an extraction coil comprising an inlet and an outlet, the inlet of the coil being connected to the outlet of the mixer, and a phase separator connected to the outlet of the coil,

wherein the mixer comprises a radial, irradiating, ultrasonic probe placed in the center of the coil.

The liquid/solid extraction device according to the invention can further have one or a plurality of the features below, taken individually or according to any technically feasible combination:
the coil is formed of a tube wound helically abound an axis so as to form turns, and wherein the ultrasonic probe extends mainly in an elongated direction, substantially parallel to the axis of the coil, and has an active length entirely contained between the turns of the coil;
the coil is placed inside a thermostatically controlled chamber;
the coil is positioned at an angle comprised between 10° and 15° with respect to the horizontal;
the ultrasonic probe comprises a plurality of fins, in particular three fins;
the phase separator comprises an Archimedes screw extending mainly along a longitudinal axis with a diameter which increases longitudinally from upstream to downstream;
the Archimedes screw is surrounded by a filter tube with a porosity comprised between 20 μm and 500 μm;
the device comprises a vacuum pump connected to the phase separator;
the device comprises a pump connected to the mixer outlet and to the coil inlet;
the coil is made of glass.

The invention further relates to a method for the continuous solid/liquid extraction of molecules of interest from a ground material produced from solid material by using an extraction device according to the invention, the mixer comprising a tank delimiting an internal volume, the method comprising introducing an extraction solvent and a solid ground material into the internal volume delimited by the mixer tank, mixing the extraction solvent and the solid ground material so as to form a homogeneous suspension, passing through the suspension extraction coil, irradiating the suspension by means of the ultrasonic probe through the coil, and passing the suspension through the phase separator, separating the suspension into a liquid phase comprising the extraction solvent enriched with molecules of interest and a solid phase comprising extraction residues.

### BRIEF DESCRIPTION OF THE DRAWINGS

Other features and advantages of the invention will appear upon reading the following description, given only as an example, and making reference to the enclosed drawings, wherein:

FIG. 1 is a schematic view of an extraction device according to the invention comprising an ultrasonic probe with radial irradiation;

FIG. 2 is a schematic view of a coil of the extraction device shown in FIG. 1;

FIG. 3 is a schematic view of the ultrasonic probe of the extraction device shown in FIG. 1; and

FIG. 4 is a schematic exploded view of a phase separator of the extraction device shown in FIG. 1.

### DETAILED DESCRIPTION OF PREFERRED EMBODIMENTS

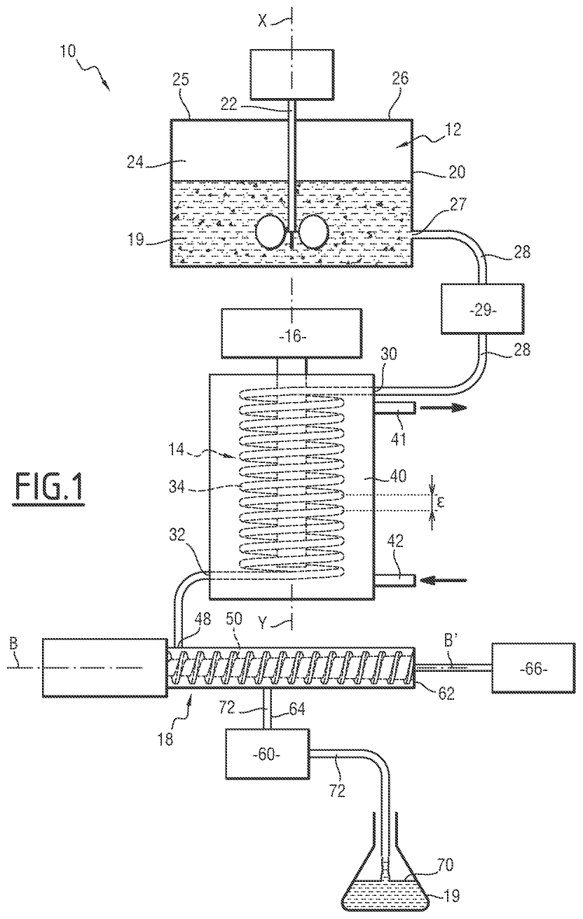

A solid/liquid extraction device 10 is shown in FIG. 1.

The solid/liquid extraction device 10 is configured for extract molecules of interest from solid material in a liquid solvent.

In particular, the solid material is a ground material of plant origin.

As an example, the plant-origin ground material is a rhizome ground of Japanese knotweed, and the molecules of interest are resveratrol, polydatin and emodin.

Of course, the solid/liquid extraction device 10 is suitable for any other plant or any other solid material which can be ground in order to extract molecules of interest therefrom.

In particular, the device 10 is suitable for the eco-extraction and the subsequent use of plant biomass, e.g. in cosmetics, pharmaceuticals, perfumery, for fine chemicals and in nutraceuticals.

With reference to FIG. 1, the device 10 includes a mixer 12, an extraction coil 14, an ultrasonic probe 16 and a phase separator 18.

Hereinafter, the terms “upstream” and “downstream” and the terms “inlet” and “outlet” are used with reference to the normal directions of flow of the fluids inside the device.

The mixer 12 is suitable for receiving the solid material and the solvent and for forming a homogeneous suspension 19.

The mixer 12 comprises a tank 20 and a mechanical stirrer 22 immersed in the tank 20.

The tank 20 delimits an internal volume 24.

The tank 20 extends mainly about an axis X and preferentially has a cylindrical shape with a flat bottom.

Such an arrangement can be used for mixing the suspension while avoiding sedimentation, i.e. preventing the ground plant material from being flattened at the bottom of the tank 20.

In a variant, the tank 20 has a cylindrical shape with a rounded bottom, or a flared, frustoconical, parallelepipedal shape, or any other possible shape.

The tank 20 is preferentially made of plastic material.

In a variant, the tank 20 is made of stainless-steel resistant to corrosive chemicals.

The internal volume 24 has a capacity comprised e.g. between 1 L and 1000 L, in particular between 1 L and 20 L.

The mixer 12 comprises a solvent inlet 25 and a solid material inlet 26 distinct from the solvent inlet 25, and an outlet 27 for the suspension 19.

In a variant, the solvent inlet 25 and the solid material inlet 26 are merged.

The mechanical stirrer 22 can rotate about an axis X′, which is vertical herein.

Preferentially, the mechanical stirrer 22 and the tank 20 are coaxial.

The mechanical stirrer 22 is chosen so that as plunge as deep as possible into the interior volume 24 of the tank 20 in order to limit as much as possible the decanting of the solid material and to ensure the best possible homogeneity of suspension.

The mechanical stirrer 22 is e.g. a blade propeller, herein a three blade propeller.

The speed of rotation of the mechanical stirrer 22 is suited to the viscosity of the suspension. Said speed is generally comprised between 60 and 3600 rotations per minute.

The outlet 27 of the mixer 12 is e.g. the end of a pipe 28 immersed in the tank 20.

The pipe 28 is e.g. made of silicone.

In a variant, the pipe 28 is a stainless-steel tube resistant to corrosive chemicals.

Hereinafter, the term “diameter” refers to the maximum extent of the pipe or of a tube considered in a transverse plane, i.e. perpendicular to the central axis of the pipe or of the tube. The above concerns e.g. the diameter of a circle in the case where the cross-section of the pipe or of the tube is circular or the diagonal of a rectangle in the case where the cross-section of the pipe or of the tube is rectangular.

The pipe 28 preferentially has a constant diameter throughout the length thereof.

The pipe 28 has e.g. a diameter of 8 mm and a length of 20 cm to 60 cm.

The diameter of the pipe 28 can of course vary and is suitable for the other elements of the device 10 and for the solid material used for extraction.

The same applies for the length of the pipe 28.

The outlet 27 of the mixer 12 is connected to the coil 14, e.g. by means of the pipe 28.

Advantageously, the extraction device 10 comprises a pump 29 between the outlet 27 of the mixer 12 and the coil 14.

The pump 29 takes the suspension 19 from the internal volume 24 of the tank 20 and conveys same to the coil 14.

The pump 29 is e.g. a peristaltic pump.

The adjustment of the pump 29 makes it possible to control the flow of the suspension 19 which enters the coil 14, and consequently the extraction time.

The flow rate is e.g. comprised between 1 L.h⁻¹ and 15 L.h⁻¹, more particularly herein between 3 L.h⁻¹ and 9 L.h⁻¹.

For the residence time of the suspension 19 in the coil 14 to be sufficient and for the extraction to be as efficient as possible, the flow rate should not be too high.

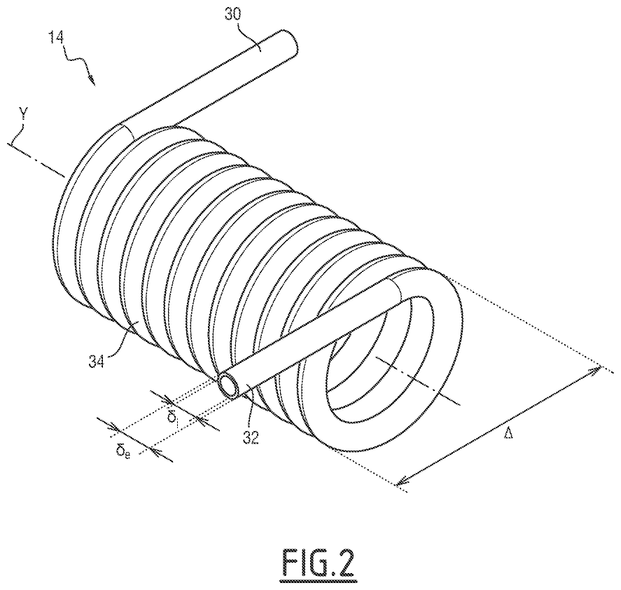

An example of coil 14 of the device 10 is shown in FIG. 2.

The extraction of molecules of interest from solid material takes place in the coil 14.

The coil 14 comprises an inlet 30 and an outlet 32.

The inlet 30 of the coil 14 is connected to the outlet 27 of the mixer 12.

The coil 14 consists of a tube 34 wound helically or spirally about an axis Y.

The tube 34 has a tube inner diameter δi and a tube outer diameter δe.

The coil 14 has a number of turns, an turn outer diameter Δ measured from the center of the turn to the outer rim of the turn with respect to the center, and a distance ε between two consecutive turns measured along the Y axis of the coil 14 as defined.

Preferentially, the tube inner diameter δi, the tube outer diameter δe, the turn outer diameter Δ and the distance ε between two consecutive turns are constant over the entire length of the coil 14 taken along the axis Y of the coil 14.

The turn outer diameter Δ is determined so as to preserve a sufficient receiving space for the ultrasonic probe 16, yet minimum in order to optimize the ultrasonic power applied.

In particular, the tube 34 has a tube inner diameter δi comprised between 0.5 cm and 3 cm and in particular equal to 1.4 cm and a tube outer diameter δe comprised between 0.8 cm and 3.8 cm and in particular equal to 1.8 cm.

In particular, the coil 14 comprises more than ten turns, in particular thirteen turns, with an inner diameter comprised between 8 cm and 20 cm depending on the diameter of the probe 16 and in particular equal to 13.5 cm.

The distance ε between two consecutive turns is also set so as to limit the reflection of the ultrasonic waves and to increase the efficiency of the ultrasonic probe 16.

In particular, the distance ε between two consecutive turns is comprised between 0.2 cm and 1 cm and in particular equal to 0.3 cm.

The tube 34 has e.g. a capacity comprised between 50 mL and 9 L and in particular equal to 880 mL.

Adjusting the number of turns or using coil extensions makes it possible to play on the extraction time. Indeed, the greater the number of turns, within the limit of the length of the ultrasonic probe 16 taken along the Y axis of the coil 14 and depending on the power of said ultrasonic probe 16 as will be described hereinafter, the longer the extraction time, the better the extraction yield.

The coil 14 is preferentially made of glass.

The above allows for a limited reflection and thus a better efficiency of the ultrasonic waves.

Advantageously, with reference to FIG. 1, the extraction device 10 comprises a thermostatically controlled chamber 40 inside which the coil 14 is placed.

The above makes it possible to control the extraction temperature depending on the molecules to be extracted.

According to a first embodiment, the coil 14 is positioned vertically in the thermostatically controlled chamber 40, as is the case in FIG. 1, so that the inlet 30 of the coil 14 is placed at a height greater than the height of the outlet 32, and that the suspension 19 flows through the coil 14, pushed by gravity.

The floor space requirement of the device 10 is thus limited.

In a variant, the coil 14 is positioned so as to form an angle comprised between 10° and 15° with respect to the horizontal.

The above makes it possible to control as much as possible, the residence time of the suspension 19 inside the coil 14 by limiting the effects due to gravity.

The thermostatically controlled chamber 40 is typically a container receiving a heat-transfer fluid and coupled to a temperature control system.

The heat-transfer fluid is e.g. a mixture of water and ethylene glycol, each at 50% by volume of the total volume of the heat-transfer fluid.

The temperature regulation system is provided e.g. by a compressor making it possible to regulate the temperature of the heat-transfer fluid and to keep same substantially constant.

Depending on the solid material and on the molecules of interest to be extracted, the temperature is set between 5° C. and 75° C.

The thermostatically controlled chamber 40 comprises an inlet 41 and an outlet 42 for the heat-transfer fluid.

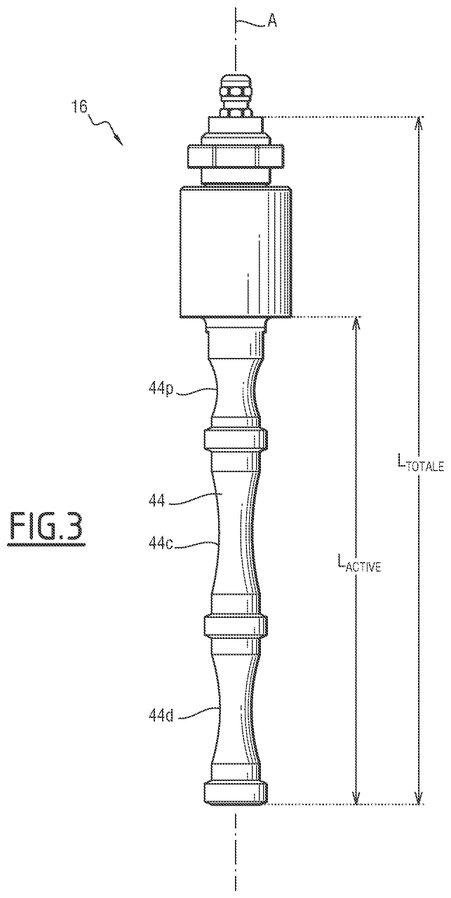

The extraction is assisted by the radial emission ultrasonic probe 16 illustrated in FIG. 3.

The ultrasonic probe 16 is immersed in the thermostatically controlled chamber 40.

The ultrasonic probe 16 is positioned at the center of the coil 14.

The ultrasonic probe 16 extends mainly along a direction of elongation A.

Preferentially, the direction of elongation A is substantially parallel to the axis Y of the coil 14 when the ultrasonic probe 16 is in place.

The ultrasonic probe 16 is e.g. at a maximum radial distance of less than 5 cm depending on the diameter of the probe, in particular 3 cm from each turn of the coil 14.

As can be seen in FIG. 3, the ultrasonic probe 16 has a total length Lₜₒₜₐₗ measured along the direction of elongation A comprised between 25 cm and 35 cm.

The ultrasonic probe 16 has an active length L_{active} comprised between 20 cm and 30 cm.

Active length L_{active} refers to the length of the ultrasonic probe 16 on which the ultrasonic probe 16 is apt to emit ultrasounds.

In particular, the ultrasonic probe 16 has an active length L_{active} equal to 24.2 cm.

The active length L_{active} of the ultrasonic probe 16 is advantageously entirely contained between the turns of the coil 14 and coaxially with respect to the coil 14.

The ultrasonic probe 16 comprises a plurality of fins 44.

“Fin” refers to a rod extending between two ends and having a flared shape toward each of the two ends.

In particular, the ultrasonic probe 16 comprises three fins 44, a proximal fin 44p, a central fin 44c and a distal fin 44d from upstream to downstream of the coil 14.

Each fin 44 has e.g. a maximum diameter comprised between 1 cm and 10 cm.

In particular, each fin 44 has a maximum diameter equal to 3 cm and a minimum diameter equal to 1.6 cm.

The central fin 44c and the distal fin 44d are identical.

The proximal fin 44p has a length less than the length of the central fin 44c or of the distal fin 44d measured along the direction of elongation A of the ultrasonic probe 16.

The electrical power of the ultrasonic probe 16 is comprised between 75 W and 750 W.

The adjustment of the electrical power makes it possible to optimize the extraction efficiency of the different targeted molecules.

The frequency of the ultrasonic probe 16 is comprised between 20 and 80 kHz.

The continuous ultrasonic irradiation increases the transfer of molecules of interest from the solid material to the solvent, depending on the residence time inside the coil 14 and on the acoustic power, while maintaining the fluidity and homogeneity of the suspension 19.

The positioning of the ultrasonic probe 16 at the center of the coil 14 is such that no direct contact is possible between the ultrasonic probe and the solid material suspended in the extraction solvent, thus limiting any risk of contamination of the suspension.

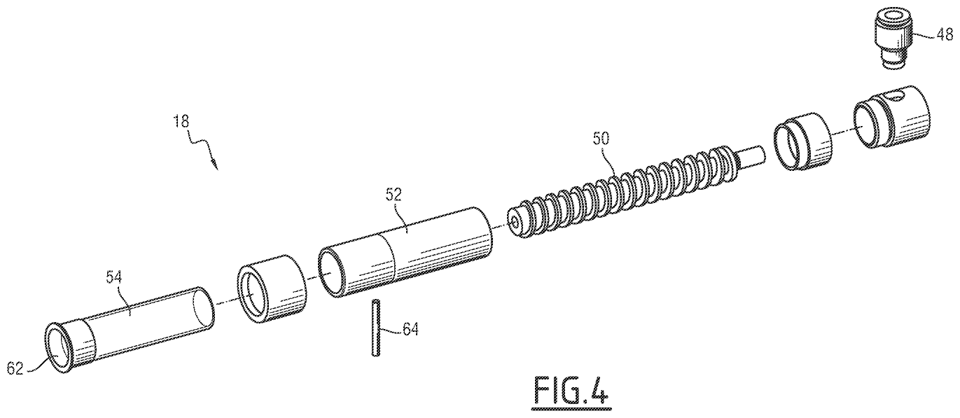

The outlet 32 of the coil 14 is connected to the phase separator 18 illustrated in FIG. 4.

The phase separator 18 comprises an inlet 48 through which the suspension 19 enters after the passage thereof through the coil 14.

The phase separator 18 comprises e.g. an Archimedes screw 50.

The Archimedes screw 50 extends mainly along a longitudinal axis B-B′.

The axis B-B′ of the Archimedes screw 50 has a diameter which advantageously increases from upstream to downstream.

The above makes it possible to compact the suspension as same flows through the Archimedes screw 50 and thus to recover a maximum of solvent enriched with molecules of interest extracted from the solid material.

The Archimedes screw 50 has a maximum diameter comprised e.g. between 1 cm and 50 cm.

In particular, the Archimedes screw 50 has a maximum diameter equal to 25 mm.

The Archimedes screw 50 has a length, as measured along the axis B-B′ thereof, comprised e.g. between 10 cm and 200 cm.

In particular, the Archimedes screw 50 has a length equal to 15 cm.

The Archimedes screw 50 is advantageously surrounded by a filter tube 52.

The tube 52 is e.g. made of a stainless steel wire cloth.

The tube 52 has a porosity comprised between 20 μm and 500 μm.

In particular, the tube 52 has a porosity substantially equal to 50 μm.

The Archimedes screw 50 and the tube 52 are advantageously placed in a tubular jacket 54.

The tubular jacket 54 is made e.g. of plexiglass material.

The jacket 54 has a diameter comprised e.g. between 2.5 cm and 50 cm.

In particular, the tubular jacket 54 has a diameter equal to 3.5 cm.

The tubular jacket 54 has a length measured along the axis B-B′ of the Archimedes screw 50, e.g. between 10 cm and 200 cm.

In particular, the tubular jacket 54 has a length equal to 15 cm.

The Archimedes screw 50 is advantageously actuated by an electric motor controlled by an adjustable-speed drive comprised between 1 and 100 rotations per minute.

The motor has e.g. a maximum permissible power of 15 V and 1 A.

The moisture of the solid recovered at the outlet is dependent on the speed of rotation of the Archimedes screw 50. If the Archimedes screw 50 rotates too quickly, the material is not drained long enough before being expelled at the outlet.

With reference to FIG. 1, the device 10 advantageously comprises a vacuum pump 60 connected to the phase separator 18.

The vacuum pump 60 is e.g. a peristaltic pump.

The vacuum pump 60 is preferentially a diaphragm pump equipped with an electronic pressure gage.

The vacuum pump 60 is e.g. configured so that the pressure measured in the Archimedes screw 50 is comprised between 100 mbar and 900 mbar.

In particular, the vacuum pump 60 is configured so that the pressure measured in the Archimedes screw 50 is equal to 800 mbar.

Too high a vacuum is not desirable, because same would block the Archimedes screw 50.

The phase separator 18 comprises a solid phase outlet 62 downstream of the Archimedes screw 50 and a liquid phase outlet 64 comprising the solvent enriched with molecules of interest.

The liquid phase outlet 64 is advantageously diametrically opposite the inlet 48 of the phase separator 18.

The layout of the phase separator 18 with the vacuum pump 60 makes it possible to recover a maximum of enriched solvent without solid material and to eliminate a cake of almost dry solid extraction residue which can be subsequently recycled.

The phase separator 18 allows solid and liquid phases to enter simultaneously. The discharge phenomena observed with backflow commercial devices are thus prevented.

The device 10 advantageously comprises a tank 66 for receiving solid phase in the vicinity of the outlet 62 of the phase separator 18.

The device 10 advantageously further comprises a decanter 70 connected to the liquid phase outlet 64.

The decanter 70 is used for separating by gravity, the fine particles remaining in the liquid phase.

The decanter 70 is e.g. an Erlenmayer flask.

The solvent enriched with molecules of interest obtained at the outlet of the phase separator 18 is e.g. conveyed via the vacuum pump 60 to the decanter 70.

The decanter 70 is e.g. connected, by a tube 72, to the outlet 64 for the solvent enriched with molecules of interest.

The tube 72 is e.g. made of silicone.

The vacuum pump 60 has the double advantage of creating a vacuum and conveying the liquid phase to the decanter 70. For the vacuum of the vacuum pump 60 to be effective, the tube 72 has to be as rigid as possible.

The phase separator 18 and the vacuum pump 60 make it possible to obtain a clear liquid solution very quickly, typically in less than an hour.

The device 10 according to the invention can be used for continuously extracting and separating a liquid phase containing the solvent enriched with molecules of interest from the solid phase obtained from ground solid material.

The ultrasonic probe 16 with radial irradiation is not in direct contact with the suspension 19, limiting any risk of contamination. The acoustic power adjustment makes it possible to optimize the extraction efficiency of the different target molecules of interest.

The device has the further advantage of being compact, movable and usable at high flow rates.

Furthermore, many parameters can be modulated depending on the molecules of interest which are to be extracted: the solid/liquid ratio of the suspension 19 by setting the speed of rotation of the Archimedes screw 50 feeding the solid material into the mixer 12, and the flow rate of the pump entraining the solvent into the mixer 12, the extraction temperature by setting the temperature of the thermostatically controlled chamber 40, the extraction time by setting the flow rate of the pump 29 entraining the suspension 19 to the inlet 30 of the coil 14 or by the length of the coil 14.

The device 10 allows the consumption of solvents and of energy to be limited, and does not require consumables, such as filters, for working, and therefore allows the operating costs to be limited.

A method for extracting molecules of interest from solid material using the extraction device 10 will now be described.

An extraction solvent and a ground solid material are introduced into the internal volume 24 delimited by the tank 20 of the mixer 12.

The extraction solvent is advantageously a mixture of water and ethanol.

In a variant, the extraction solvent is a solvent or a mixture of solvents of different nature, technically conceivable for performing a solid/liquid extraction.

The proportions of the mixture of water and ethanol are comprised between 1:99 and 99:1 by volume with respect to the total volume of solvent.

The extraction solvent is e.g. prepared upstream and then conveyed to the internal volume 24 of the tank 20 by a metering pump.

In a variant, the extraction solvent is produced directly at the inlet of the mixer 20 by means of a binary pump.

The solid material is e.g. provided in the form of a dry powder with a particle size distribution comprised between 0.2 mm and 1 mm.

The solid material is e.g. supplied by means of an Archimedes screw with set speed of rotation so as to maintain a set concentration in the mixer 12.

The extraction solvent and the solid material are fed continuously into the mixer 12 so as to form a suspension 19.

The adjustment of the speed of rotation of the Archimedes screw and of the flow rate of the solvent pump at the inlet of the mixer 12 allows the solid/liquid ratio of the suspension 19 to be varied as required.

A homogeneous suspension 19 is obtained. The mass concentration of the ground material of plant origin in the extraction solvent is e.g. comprised between 100 g.L⁻¹ and 150 g.L⁻¹.

The suspension 19 is entrained from the outlet 27 of the mixer 12 to the inlet 30 of the coil 14.

Advantageously, the pump 29 sets the entrainment flow rate of the suspension 19. The flow rate is e.g. comprised between 1 L.h⁻¹ and 15 L.h⁻¹, more particularly between 3 L.h⁻¹ and 9 L.h⁻¹.

The flow rate is set so as to control the average residence time of the suspension 19 inside the coil 14 in order to achieve the maximum extraction efficiency.

Advantageously, the residence time of the suspension inside the coil 14 is comprised between 30 seconds and 30 minutes, preferentially between 5 minutes and 30 minutes.

The thermostatically controlled chamber 40 is set to a fixed temperature in order to control the extraction temperature. The temperature of the thermostatically controlled chamber 40 is e.g. comprised between 5° C. and 75° C.

The ultrasonic probe 16 is connected to a generator enabling same to irradiate the contents of the coil 14 at a frequency comprised between 20 kHz and 80 kHz, in particular at 20 kHz.

Under the effect of temperature and ultrasonic irradiation, the molecules of interest are extracted from the solid material by the extraction solvent.

The suspension 19 in the solvent enriched with molecules of interest is conveyed from the outlet 32 of the coil 14 to the phase separator 18.

The inlet flow-rate of the suspension into the phase separator 18 is equal to the entrainment flow-rate of the suspension from the mixer 12 to the coil 14.

The phase separation is preferentially carried out by vacuum pressing through the Archimedes screw 50, pushing the spun solid phase outwards from the Archimedes screw 50.

The liquid phase comprising the enriched solvent is discharged more efficiently by means of the vacuum suction provided by the vacuum pump 60.

The vacuum pump 60 is e.g. set so that the pressure measured in the Archimedes screw 50 is comprised between 100 mbar and 900 mbar.

In particular, the vacuum pump 60 is set so that the pressure measured in the Archimedes screw 50 is equal to 800 mbar.

The liquid phase having crossed through the tube 52 surrounding the Archimedes screw 50 is recovered in the decanter 70.

By means of the method described according to the invention, the solvent enriched with molecules of interest is recovered completely free of solid particles with a particle size greater than 20 μm.

If need be, the addition of a paper cartridge removes solid particles with a particle size greater than 5 μm.

Such filtration step allows the solvent enriched with molecules of interest in e.g. a purification process to be used directly.

Overall extraction yields are improved by combining pressing by means of an Archimedes screw and vacuum suction for collecting the maximum amount of solvent enriched with molecules of interest.

Furthermore, the solid phase is recovered spun and separated from the solvent and thus remains available for being subsequently recycled.

The method described in the invention can be used for simplified extraction, filtration and separation by using one device.

After use, the cleaning of the device 10 is advantageously easy.

A cleaning liquid is taken from the tank 20 of the mixer 12 and conveyed into the coil 14 while maintaining the ultrasonic irradiation for the time required for all the solid material to be removed from the coil 14.

The cleaning liquid is e.g. demineralized water.

To clean from the phase separator 18, the fine particles which could have been deposited on the tube 52 surrounding the Archimedes screw 50, while maintaining the rotation of the Archimedes screw and the suction by the vacuum pump 60.

The cleaning liquid is e.g. demineralized water.

In order to clean the Archimedes screw 50 from solid residues, a cleaning liquid is conveyed while maintaining the rotation of the Archimedes screw and cutting off the suction produced by the vacuum pump 60.

The cleaning liquid is e.g. demineralized water.

The method is thus suitable for any type of solid/liquid extraction involving a solid material the particle size distribution of which is comprised between 0.2 mm and 1 mm, and an extraction solvent in order to enrich the solvent with molecules of interest and to continuously separate the solvent from the solid phase.

### Example of Implementation of the Device According to the Invention

Operating Conditions:
Solvent EtOH/H₂O (6/4 v/v).
Particle size distribution of the ground plant 200 μm<G<1 mm.
mass concentration of the ground material of plant origin suspended in the solvent: 100 g.L⁻¹.
Peristaltic pump flow-rate between the mixer and the coil: 50 mL.min⁻¹, 3 L.h⁻¹.
Amplitude set on the ultrasonic generator: 100% of the maximum electrical power (750 W).
Archimedes screw: 13 V, 0.8 A.

1—Tank Containing the Suspension

The suspension was prepared with Japanese knotweed rhizome ground to a particle size distribution comprised between 200 μm and 1 mm. The particle size was controlled by means of stainless-steel sieves used on the dry powder.

If the powder contained particles with a particle size distribution of less than 200 μm, the filtration system very quickly became clogged.

2—Mechanical Stirrer

The mechanical stirrer was set at a speed of rotation 500 rotations per minute. The three-blade propeller was plunged as deep as possible into the tank so as to limit the settling of the solid material as much as possible and to provide the best possible homogeneity.

3—Peristaltic Pump

The peristaltic pump was set for a minimum flow rate of 50 mL.min⁻¹ (3 L.h⁻¹), i.e. a residence time comprised between 60 seconds and 90 seconds inside the coil within the framework of the tests run. Below said speed, there was a risk of agglomeration of the suspension and of clogging inside the pipe bringing the suspension from the mixer to the coil. The pipe had a diameter of 8 mm.

4—Thermostatically Controlled Enclosure

The temperature of the enclosure was controlled by means of a Minichiller® type circulation cooler from Huber e.g., or of an oil bath heater.

5—Radial Ultrasonic Probe

The radial ultrasonic probe was positioned at the center of the glass coil and set at a frequency of 20 kHz. The active length of the ultrasonic probe was entirely contained within the turns of the coil, coaxially with the coil.

The probe was 3 cm away from each side of the coil. The maximum power was 750 W and could be set from 10 to 100% of the rated power thereof. The radial ultrasonic probe and the associated generator were e.g. of the SinapTec make (Ultrasonic Processor NextGen Lab750).

During the tests, the probe was used to the maximum of the capacity thereof without any known problem either at the coil or at the thermostatically controlled enclosure.

6—Coil

The glass coil consisted of 13 turns with an outer diameter of 13.5 cm. The coil tube had an inner diameter of 1.4 cm and a total length of 5.7 m, i.e. a capacity of 880 mL. The coil was placed vertically, the inlet being placed higher than the outlet, so that the suspension flowed by gravity.

7—Archimedes Screw for Solid/Liquid Separation

The suspension collected at the outlet of the coil was continuously separated into two phases: liquid and solid. The suspension entered the continuous filtration system vertically. The liquid phase was extracted under vacuum from a tube diametrically opposite the inlet of the suspension. The Archimedes screw surrounded by a stainless-steel tubular grid with a porosity of 50 μm allowed the dried solid phase to be conveyed as the liquid phase was sucked in. The motor actuating the Archimedes screw was supplied with a current of 13 V and 0.8 A. The acceptable power for the motor was directly set on the power supply unit. The moisture of the solid recovered at the outlet depended on the speed of rotation of the screw. If the screw rotated too quickly, the material was not spun sufficiently before being expelled at the outlet. Under the test conditions, a setting to 13 V allowed an optimum speed for a flow rate of 3 L.h⁻¹ to be achieved. Below 13 V, the solid could compact in the screw and block the device.

The values given here are indicative, a change in the pump flow rate or in the dilution and in the particle size distribution parameters would directly affect such values.

8—Recovery of the Solid

The almost dry solid phase was recovered in a tank at the outlet of the screw.

9—Decanter—Recovery of the Liquid Phase

The liquid phase obtained at the outlet of the phase separator was conveyed via a peristaltic pump to an Erlenmeyer flask allowing fine particles with a particle size of less than 50 μm to be decanted.

10—Peristaltic Pump for the Recovery of the Liquid Phase.

In order to ensure a better spinning of the solid, the phase separator was placed under vacuum. The vacuum was provided by the peristaltic pump mentioned hereinabove which had the double advantage of creating a vacuum and of conveying the liquid phase to the decanter.

1. A device for continuous solid/liquid extraction comprising at least, from upstream to downstream:
a mixer comprising an inlet and an outlet an extraction coil comprising an inlet and an outlet, the inlet of the coil being connected to the outlet of the mixer, and a phase separator connected to the outlet of the coil,
wherein the device comprises an ultrasonic probe with radial irradiation placed in the center of the coil.

2. The device according to claim 1, wherein the coil is formed of a tube extending helically about an axis so as to form turns, and wherein the ultrasonic probe extends mainly along an elongation direction, substantially parallel to the axis of the coil and has an active length entirely contained between the turns of the coil.

3. The device according to claim 1, wherein the coil is located within a thermostatically controlled chamber.

4. The device according to claim 1, wherein the coil is positioned by forming an angle comprised between 10° and 15° with the horizontal.

5. The device according to claim 1, wherein the ultrasonic probe has a plurality of fins.

6. The device according to claim 1, wherein the phase separator comprises an Archimedes screw extending mainly along a longitudinal axis having a diameter which increases longitudinally from upstream to downstream.

7. The device according to claim 6, wherein the Archimedes screw surrounded by a filter tube having a porosity comprised between 20 μm and 500 μm.

8. The device according to claim 1, comprising a vacuum pump connected to the phase separator.

9. The device according to claim 1, comprising a pump connected to the outlet of the mixer and to the inlet of the coil.

10. The device according to claim 1, wherein the coil is made of glass.

11. A method of continuous solid/liquid extraction of molecules of interest from a ground material produced from solid material by using an extraction device according to claim 1, the mixer comprising a tank delimiting an internal volume, the method comprising introducing an extraction solvent and a solid ground material into the internal volume delimited by the tank of the mixer, mixing the extraction solvent and the solid ground material so as to form a homogeneous suspension, passing through the coil for extracting the suspension, irradiating the suspension by means of the ultrasonic probe through the coil, and passing the suspension through the phase separator, separating the suspension into a liquid phase comprising the extraction solvent enriched with molecules of interest and a solid phase comprising extraction residues.

12. The device according to claim 2, wherein the coil is located within a thermostatically controlled chamber.

13. The device according to claim 2, wherein the coil is positioned by forming an angle comprised between 10° and 15° with the horizontal.

14. The device according to claim 3, wherein the coil is positioned by forming an angle comprised between 10° and 15° with the horizontal.

15. The device according to claim 2, wherein the ultrasonic probe has a plurality of fins.

16. The device according to claim 3, wherein the ultrasonic probe has a plurality of fins.

17. The device according to claim 4, wherein the ultrasonic probe has a plurality of fins.

18. The device according to claim 2, wherein the phase separator comprises an Archimedes screw extending mainly along a longitudinal axis having a diameter which increases longitudinally from upstream to downstream.

19. The device according to claim 3, wherein the phase separator comprises an Archimedes screw extending mainly along a longitudinal axis having a diameter which increases longitudinally from upstream to downstream.

20. The device according to claim 4, wherein the phase separator comprises an Archimedes screw extending mainly along a longitudinal axis having a diameter which increases longitudinally from upstream to downstream.
